(19) United States
(12) Patent Application Publication
(10) Pub. No.: US 2026/0086555 A1
(43) Pub. Date: Mar. 26, 2026

(54) 3D NAVIGATION OF A MAGNETIC ROTATING SWIMMER

(71) Applicant: University of Houston System, Houston, TX (US)

(72) Inventors: Julien Leclerc, Houston, TX (US); Yitong Lu, Houston, TX (US); Aaron Becker, Houston, TX (US)

(21) Appl. No.: 19/339,417

(22) Filed: Sep. 25, 2025

Related U.S. Application Data
(60) Provisional application No. 63/699,043, filed on Sep. 25, 2024.

(51) Int. Cl. G05D 1/242; G05D 1/46; G05D 109/30; G05D 111/20

(52) U.S. Cl. CPC G05D 1/242; G05D 1/46; G05D 2109/30; G05D 2111/20

(57) ABSTRACT

A system for 3D navigation of a magnetic rotating swimmer includes a magnetic manipulator with coils defining a 3D workspace, a probe positioned to obtain a 2D image of an imaging plane from the 3D workspace, a robotic arm coupled to the probe and to move the probe and the imaging plane, and a processor network in communication with instructions to operate the magnetic manipulator to move the swimmer on a path in the 3D workspace, to obtain 2D images with the probe while the swimmer is moving in the 3D workspace, to detect the swimmer in the images, and to start, in response to detecting the swimmer, a closed-loop control of the robotic arm to move the probe to track the swimmer movement in the 3D workspace.

### STATEMENT REGARDING FEDERALLY SPONSORED RESEARCH OR DEVELOPMENT

This invention was made with government support under contract/grant number CPS-1931884 awarded by the National Science Foundation (NSF). The government has certain rights in the invention.

### TECHNICAL FIELD

This disclosure relates in general to the field of biocompatible miniature magnetic robot swimmers, and more particularly, but not by way of limitation, to optimized geometries to mitigate becoming stuck in a thrombus.

### BACKGROUND

This section provides background information to facilitate a better understanding of the various aspects of the disclosure and is not an admission of prior art.

Miniature, or millimeter scale, magnetic rotating swimmers are untethered machines containing magnetic materials. An external rotating magnetic field produces a torque on the swimmers to make them rotate. Magnetic rotating swimmers have propeller fins that convert the rotating motion into forward propulsion. This type of robot has been shown to have potential applications in the medical realm.

### SUMMARY

An example system for 3D navigation of a magnetic rotating swimmer includes a magnetic manipulator with coils defining a 3D workspace, the magnetic manipular is operable to steer the swimmer in the 3D workspace, a probe positioned to obtain a 2D image of an imaging plane from the 3D workspace, a robotic arm coupled to the probe and operable to move the probe and the imaging plane, and a processor network in communication with the magnetic manipulator, the probe, and the robotic arm, the processor network including instructions to operate the magnetic manipulator to move the swimmer on a path in the 3D workspace, to obtain 2D images with the probe while the swimmer is moving in the 3D workspace, to detect the swimmer in the images, and to start, in response to detecting the swimmer, a closed-loop control of the robotic arm to move the probe to track the swimmer movement in the 3D workspace.

An example of a method for 3D navigation of a magnetic rotating swimmer in a 3D workspace includes positioning the swimmer in a 3D workspace defined by coils of a magnetic manipulator, positioning a probe coupled to a robotic arm to obtain a 2D image from the 3D workspace along an imaging plane, navigating the swimmer along a 3D path in the 3D workspace with the magnetic manipulator, obtaining the 2D image along the imaging plane, processing the 2D image to detect the swimmer in the imaging plane, and initiating, in response to detecting the swimmer in the imaging plane, a closed-loop control of the robotic arm to move the probe to track the swimmer movement along the 3D path.

This summary is provided to introduce a selection of concepts that are further described below in the detailed description. This summary is not intended to identify key or essential features of the claimed subject matter, nor is it intended to be used as an aid in limiting the scope of claimed subject matter.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion. As will be understood by those skilled in the art with the benefit of this disclosure, elements and arrangements of the various figures can be used together and in configurations not specifically illustrated without departing from the scope of this disclosure.

FIG. 1 is a schematic illustration of an example system for three-dimensional navigation of an untethered magnetic rotating swimmer according to one or more aspects of the disclosure.

FIG. 2 illustrates a robot arm mounted ultrasound probe and reference frames related to the probe images according to one or more aspects of the disclosure.

FIG. 3 is schematic block diagram of a system for three-dimensional navigation of an untethered magnetic rotating swimmer according to one or more aspects of the disclosure.

FIG. 4 illustrates an example of a millimeter scale magnetic rotating swimmer according to one or more aspects of the disclosure.

FIG. 5 is a section view of a millimeter scale magnetic rotating swimmer according to one or more aspects of the disclosure.

FIG. 6A is schematic block diagram of a closed-loop system according to one or more aspects of the disclosure.

FIG. 6B is a continuation of the schematic block diagram of FIG. 6A according to one or more aspects of the disclosure.

FIG. 6C is a continuation of the schematic block diagram of FIGS. 6A and 6B according to one or more aspects of the disclosure.

### DETAILED DESCRIPTION

It is to be understood that the following disclosure provides many different embodiments, or examples, for implementing different features of various illustrative embodiments. Specific examples of components and arrangements are described below to simplify the disclosure. These are, of course, merely examples and are not intended to be limiting. For example, a figure may illustrate an exemplary embodiment with multiple features or combinations of features that are not required in one or more other embodiments and thus a figure may disclose one or more embodiments that have fewer features or a different combination of features than the illustrated embodiment. Embodiments may include some but not all the features illustrated in a figure and some embodiments may combine features illustrated in one figure with features illustrated in another figure. Therefore, combinations of features disclosed in the following detailed description may not be necessary to practice the teachings in the broadest sense and are instead merely to describe particularly representative examples. In addition, the disclosure may repeat reference numerals and/or letters in the various examples. This repetition is for the purpose of simplicity and clarity and does not itself dictate a relationship between the various embodiments and/or configurations discussed.

Miniature magnetic rotating swimmers, i.e., millimeter scale, are an attractive technology that provides an alternative approach for treating biological disorders inside the body, especially inside the vascular system. An external rotating magnetic field creates a torque on the swimmer and makes it rotate. The rotational motion can be used to disrupt a thrombus mechanically.

Disclosed are new methods and tools that enable the 3D control of a magnetic swimmer using a 2D ultrasound device attached to a robotic arm to sense the swimmer's position. An algorithm that computes the placement of the robotic arm and a controller that keeps the swimmer within the ultrasound imaging slice. The position measurement and closed-loop control have been tested experimentally. The closed-loop control method can steer a free-swimming, i.e., untethered, magnetic rotating swimmer along an arbitrary path using only data from the 2D ultrasound device for position feedback. Software modules are introduced for a 3D off-plane oscillation controller, a module calculates the proper orientation of the ultrasound probe, and a module performs a change of reference frame coordinates within the ultrasound imaging slice to the workspace frame. A spring loaded probe holding is also utilized.

Magnetic untethered robotic agents may be the next step in the evolution of minimal invasive surgery. Magnetic manipulation controls the position of a magnetic swimmer inside an environment via a magnetic field changing in strength and orientation over time. The properties of the magnetic field are controlled by a robotic system. The magnetic field generated by the magnetic manipulator is at a low frequency (less than 100 Hz), which allows it to pass through the human body. The properties of the magnetic field, coupled with the small nature of the swimmers, allow them to perform minimal invasive surgery. Previous research demonstrates these magnetic robots can be controlled in a simulated physiological environment and perform tasks, such as delivering targeted therapy and clearing blood clots.

This disclosure is for a magnetic rotating swimmer (MRS), a type of magnetic tetherless robotic agent. We target the treatment of pulmonary embolism because pulmonary arteries are deep in the body and difficult to reach using conventional catheters. The MRS is propelled and steered by a magnetic manipulator. A cylindrical magnet with radial magnetization is glued inside the swimmer. The magnetic manipulator creates a magnetic field that generates a torque on the swimmer, making it rotate. Propeller fins on the swimmer convert this rotation into a propulsive force. The magnetic manipulator can steer the swimmers in 3D because they automatically orient themselves with the axis of rotation of the magnetic field. Changing the direction of the magnetic field's rotation axis changes the swimmer's direction.

Navigation of MRS inside small vessels does not require closed-loop control of the radial position of the swimmer. Previous system steer the swimmers inside a vascular system that constrains the swimmer from moving in a local one-dimension system. Previous works have also experimentally demonstrated the control of swimmers under ultrasound guidance in restricted channels. If the diameter of the blood vessel is smaller than the length of the swimmer, the wall of the vessel guides the swimmer. However, using this method in large vessels, such as the vena cava, which has a diameter of up to 20 mm, will likely result in erratic motion of the swimmer because of wall collisions and unrestricted rotation during these collisions. Closed-loop control of the radial position of the swimmer inside of arteries will facilitate avoiding collisions with the walls of the arteries and prevent potential damage to the endothelium.

Closed-loop position control of an MRS requires position measurements. Several medical imaging methods are available. X-rays can provide high-resolution images and 3D volume reconstructions with CT scanners. However, X-rays are a form of ionizing radiation, sufficient exposure to which increases the risk of cancer. CT scanners, for example, increase the risk of cancers, and the risk increase is positively correlated with the radiation dose. Excessive exposure to X-rays by the patient would defeat the main purpose of MISs, which is to improve the outcome compared to other surgeries. MRI is another medical imaging method without the drawback of ionizing radiation. However, it uses a strong magnetic field that interacts with the MRS and prevents it from rotating. Photoacoustic imaging is yet another non-ionizing imaging method. It relies on a pulsed laser that locally heats up tissue to produce a thermoelastic expansion and ultrasound wave. These waves are detected and processed to produce an image. This method is sensitive to metallic objects, such as the permanent magnets embedded in swimmers. It however suffers from a lower penetration depth than traditional ultrasound which can also obtain images of an MRS inside a vascular environment. While other groups have demonstrated closed-loop control with X-ray imaging, ultrasound can provide position information without dangerous radiation. 3D ultrasound suffers from a low frame rate, but 2D ultrasound can provide images at relatively high frequencies. In this disclosure we take advantage of the high frame rate and non-ionizing properties of 2D ultrasound and use this method to obtain position feedback of an MRS.

Experimentally an MRS has been steered in an unconstrained cuboid volume to model control in areas where the MRS is small compared to the local physiological environment. This type of steering requires closed-loop control. In previous work, closed-loop control of an MRS in 2.5D using a non-moving 2D ultrasound device for position feedback, allowed the MRS to follow a horizontal 2D path, however, the MRS could not be steered on a 3D path.

Locating and tracking the swimmer in 3D requires coordinated control of the ultrasound probe's position and orientation. As disclosed herein, a combination of software and hardware components have enabled closed-loop 3D path following by moving an ultrasound probe with a 6-DOF robotic arm.

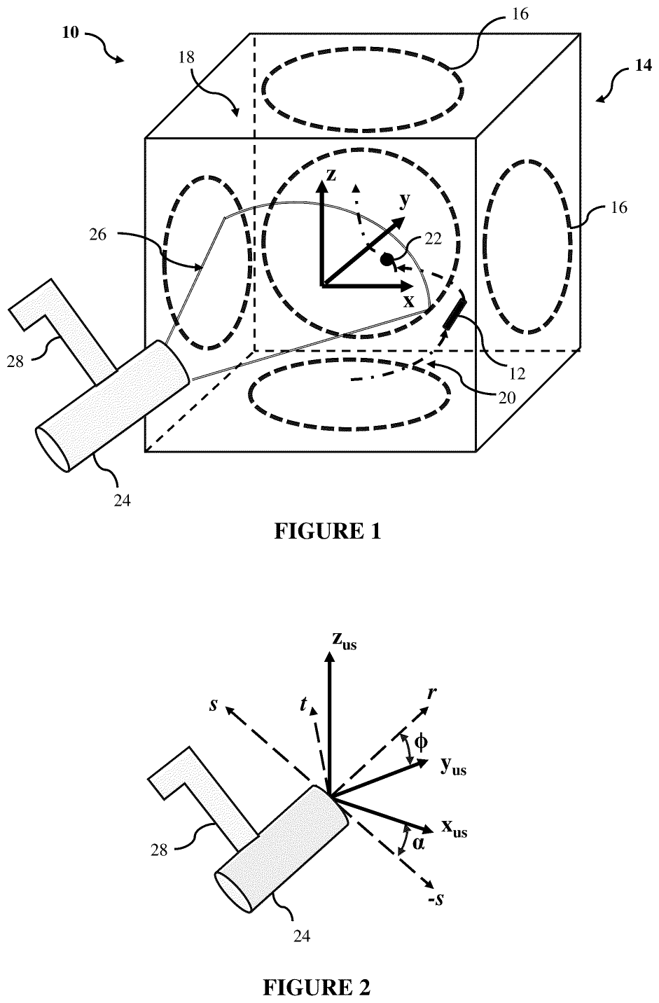

FIGS. 1 to 3 illustrate components of a system 10 for three-dimensional (“3D”) navigation of an untethered magnetic rotating swimmer (“MRS”) 12. FIG. 1 illustrates a magnetic manipulator 14, which includes electromagnetic coils 16 arranged to define a 3D workspace 18 in which MRS 12 can be steered along a 3D path 20. Path 20 may be defined by a plurality of waypoints, or locations 22. Workspace 18 has a three-axis frame of reference (x, y, z). In this example, magnetic manipulator 14 includes six coils 16 (five of which are illustrated) arranged to form a cubic workspace to move the MRS three dimensions. A probe 24 is positioned external to workspace 18 to obtain a two-dimensional (“2D”) image of an imaging plane 26 from the workspace 18. In the illustrated embodiment, probe 24 is an ultrasound probe. A robotic arm 28 is coupled to probe 24 and operable to move probe 24 and thus imaging plane 26. Robotic arm 28 has, for example, six degrees of freedom to move probe 24 such that imaging plane 26 can follow MRS 12 along 3D path 20.

FIG. 2 illustrates probe 24 and reference frames for coordinate transformations. The imaging plane 26 (FIG. 1) is along the plane of the s-axis and the r-axis (s-r plane). The probe only detects the swimmer on the imaging plane and thus the swimmer's position when detected in the image will always be at t=0. The swimmer's location within the image plane [s, r, 0] must be converted to a set of coordinates in the reference frame of the workspace [x, y, z]. An intermediate coordinate system (xᵤₛ, yᵤₛ, zᵤₛ) is used to assist the transformation of the location of the swimmer from the image plane coordinates (s, r) to the 3D workspace coordinates (x, y, z). Angle φ is the rotation angle of the image plane about the x-axis of the workspace and angle α is the rotation angle of the image plane about the y-axis. The reference frame is centered at s=0. Rotation matrices are used to convert the coordinates r and s into the workspace reference frame.

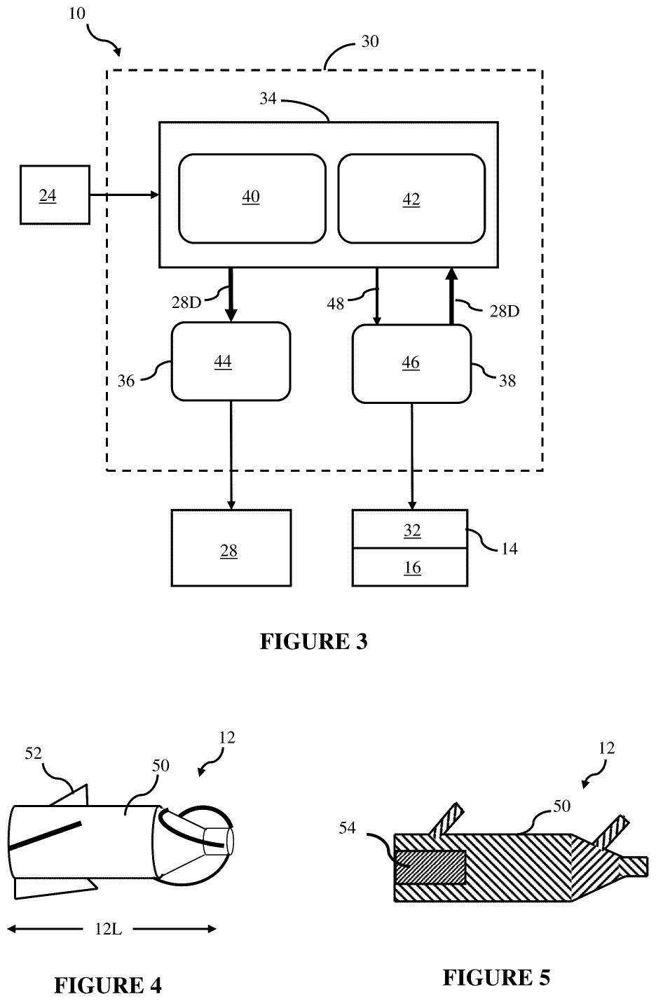

FIG. 3 schematically illustrates a system 10. System 10 includes a processor network 30 in communication with magnetic manipulator 14, probe 24, and robotic arm 28. Processor network 30 is in communication with power supply 32 to controllably energize electromagnetic coils 16 of magnetic manipulator 14. Processor network 30 includes one or more processors with operating instructions. For example, FIG. 3 illustrates processor network with a first processor 34, and second processor 36, and third processor 38. First processor 34, e.g., ultrasound processor, includes instructions 40 for ultrasound data acquisition and reconstruction and instructions 42 for MRS detection in the image and change of reference frame of the MRS location. Second processor 36, e.g., robot arm processor, includes instructions 44 to implement movement of robot arm 28 to a calculated robot arm position to locate probe 24 and the imaging plane. Third processor 38 includes instructions 46 for swimmer and probe control instructions. Robot arm position data 28D is communicated from first processor 34 to second processor 36 and from third processor 38 to first processor 34. Swimmer coordinate data 48 is communicated from first processor 34 to third processor 38

FIG. 4 illustrates an example MRS 12 and FIG. 5 is a sectional view of MRS 12 of FIG. 4. MRS 12 has a cylindrical body 50 with external fins 52 and an internal magnet 54. Body 12 has a length 12L of less than about 10 mm and in this example is about 6 mm.

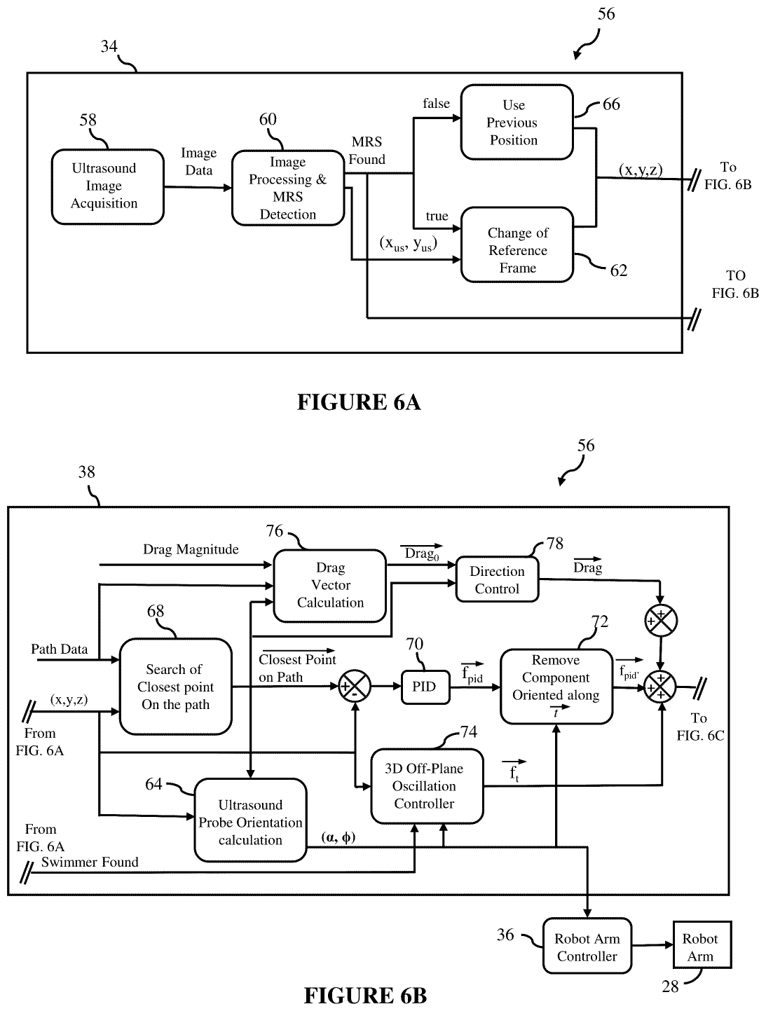

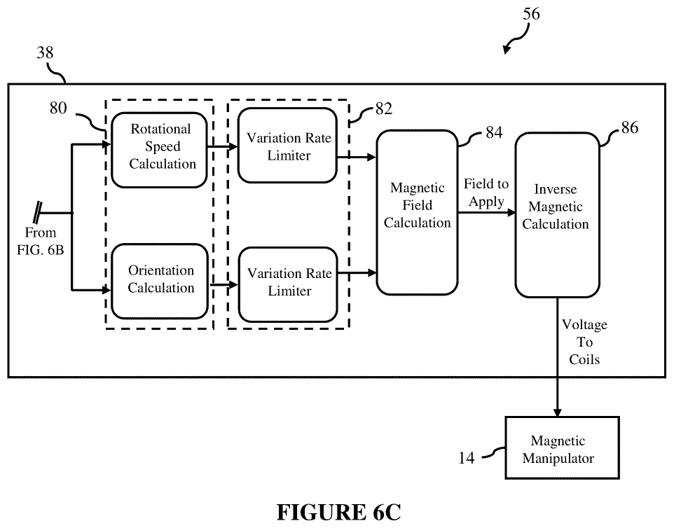

A schematic block diagram of a closed-loop system 56 according to one or more aspects of the disclosure is illustrated in FIGS. 6A-6C and described with reference to FIGS. 1 to 5. A first step 58 of the algorithm loop is to acquire an ultrasound image, which is then filtered 60 for MRS detection. If the swimmer is detected, at block 62 the swimmer's position inside the image plane is calculated and a change of reference frame function is applied to express the swimmer's position in a reference frame associated with workspace 18. This calculation requires the knowledge of the position of the robotic arm (block 64), which was calculated in the previous program loop iteration. At block 66, if the swimmer is not detected, the previous position is used. At block 68, the closest point, e.g., point 22 (FIG. 1), on path 20 is then searched, and a force component F_{PID} to apply to swimmer 12 to steer it toward the centerline of path 20 is computed from a PID controller 70. The PID controller only controls the MRS along the r and s axes, which are parallel to the ultrasound imaging slice/plane 26, and at block 72 the component along the t-axis is removed. At block 74, control over the t-axis, which is perpendicular to the imaging plane, is performed by a 3D off-plane oscillations controller (“3D-OPOC”). This module calculates a force Fₜ to apply to the swimmer to keep it inside the imaging slice.

The force magnitude needed to compensate for the drag produced by the swimmer's movement along the trajectory is a user-defined constant. It controls the velocity of the MRS. A module 76 computes the drag vector, which has the same direction as the path centerline at the point closest to the MRS's position. A module 78 changes the direction of the drag vector to make the MRS change direction when it reaches the extremities of the path, which is not closed.

At FIG. 6C, the forces to apply are then summed (block 80), and the result is sent to modules (blocks 84, 86) that calculate the magnetic field and electromagnet currents to apply. The variation rate of the magnetic field rotation axis and the swimmer's rotational speed is limited, block 82, because fast variations cause the swimmer to step out of synchronization with the magnetic field and fall to the workspace bottom.

The new orientation of the ultrasound probe is calculated in each iteration, but this information is only sent to the robotic arm every 0.4 seconds to allow the arm to finish its movement before a new movement is requested.

The ultrasound probe returns a 2D image representing the acoustic return of a nonzero-thickness imaging slice from the workspace. An equation of an imaging plane is calculated and used to determine the proper orientation of the probe. The imaging plane is the plane parallel to the imaging slice (i.e., parallel to the (r, s) plane) and at the middle slice/planes thickness, at t=0. The goal is to place the imaging plane on the path point closest to the last known position of the swimmer and parallel to the path to follow at this location. The 3D path to follow is defined by a set of waypoints. In an experiment, a total of 1,000 waypoints for a 90 mm long trajectory, which corresponds to a resolution of 0.09 mm, were used. To compute the equation of the imaging plane, the program first searches for the waypoint closest to the swimmer's last known position. The program then saves the coordinates of the waypoints just after and just before. These two points lie in the desired imaging plane. Three points are needed to define a plane. We chose to use the position of the tip of the ultrasound probe as the third point. We are free to choose the position on this point. We chose to keep the probe at the edge of the workspace at x=z=0. Coordinates of the third point of the imaging plane are therefore (0, −L_{w}/2, 0) in the (x, y, z) coordinate system where L_{w} is the length of the workspace in the y direction.

In the example, a 3D-printed checkerboard skeleton was used to calibrate the ultrasound images. The board consisted of squares with diagonals of 20 mm and was placed inside a water tank in the center of the magnetic manipulator. Ultrasound B-mode images of the skeleton were recorded, revealing the rib intersections. Images were processed to determine the physical pixel size along the r-and s-axes. A conversion factor for the s-axis is 0.295 mm/pixel with a relative error of 4 %. In the r-axis, a conversion factor is 0.265 mm/pixel with a relative error of 0.8 %. These factors are used to convert pixel coordinates into millimeter coordinates.

In the example, the swimmer's location within the ultrasound image frame [s, r, 0] is converted to a set of coordinates in the reference frame of the workspace [x, y, z]. The raw ultrasound image is 400×386 pixels. The reference frame is centered at s=0 and the pixel coordinates are converted to millimeter coordinates.

In the experimental setup, the magnetic manipulator consists of six electromagnets oriented in a cube shape and separated by 300 mm. Each electromagnet has an internal diameter of 180 mm and an external diameter of 220 mm. Each electromagnet is powered by two Kepco BOP 20-50MG power supplies connected in series. A National Instruments IC3173 industrial controller generates an analog signal to control the power supplies. Two Basler acA800 cameras are mounted on the top and right side of the magnetic manipulator to measure the swimmer's position during path-following.

The magnetic rotating swimmer 12 in the experiment has three head helices and three body helices 52 (see, FIG. 4). The bottom pitch of the helices is 16 mm, and the top pitch is 12 mm. The swimmer diameter is 2.8 mm and the length is 6 mm. These dimensions ensure the swimmer is small enough to be inserted inside a vessel yet large enough to be efficiently manufactured.

The swimmer was printed using a Formlabs resin 3D printer. Then, the swimmer is washed using ethanol inside an ANYCUBIC wash and cure station for around 6 minutes. After washing, the swimmer is placed under a UV light for 20 minutes to cure the resin and strengthen the design. One cylinder-shaped NdFeB permanent magnet (1 mm diameter, 1.5 mm length) is placed inside the swimmer. A small amount of epoxy is added at the bottom of the swimmer and ensures that the magnet stays in place.

The ultrasound system utilized in the experiment is a Verasonics Vantage 32 LE with a 64-element 1D phased array transducer. A configuration file in the form of a MATLAB script is used to set the parameters of the ultrasound acquisition. The frequency of the ultrasound probe is 3 MHz. The configured threshold of the Doppler signal is 0.2 (20 %). The ultrasound system is connected to a host controller computer. The control and data acquisition of the ultra-sound transducer is controlled via a MATLAB graphical user interface (GUI). An ultrasound gel pad (diameter 90 mm, thickness 20 mm) is placed between the probe and the wall of the water tank to reduce the acoustic impedance at this interface. Furthermore, as the probe is rotated, the extremities of the probe tip may be pushed against the gel pad, and so a spring-loaded probe holder is used to allow the probe to move back to prevent damaging the workspace.

The Verasonics Host Computer is connected to a National Instrument IC3173 through a TCP/IP connection via an ASUS AC3100 Router, see, e.g. FIG. 3. The Verasonics computer sends the MRS position to the IC3173 using this connection. A LabVIEW script in the IC3173 calculates the pose for the UR3 robot arm. The generated pose is sent via the Verasonics computer to the UR3 arm.

A program moving the robotic arm is implemented on the UR3 robot arm computer. The program is a combination of its visual scripting language and URscript commands. On startup, a connection is established to the Verasonics host computer hosting the TCP server. The robot arm waits for a set of arm pose coordinates sent as a string variable. Robot arm pose coordinates are composed of linear translation values (in millimeters) and rotational values (in radians) along the x, y, and z axes. Individual coordinates are extracted from the string and placed into variables. The linear translation coordinates are converted from meters to millimeters. A pose variable is generated, and the robot arm moves to this specified pose in a timeframe of 400 ms. The moveL function inside the URScript program moves the arm to the correct position.

### Experimental Results

We first validated the proposed swimmer position measurement method by making the swimmer follow a pre-defined 3D path in a water tank using camera feedback to close the control loop. The ultrasound transducer was attached to the robotic arm, which set the pose of the transducer. The position in the workspace reference frame was calculated using the method introduced above. We compared the position information measured using cameras and ultrasound. An offset was manually tuned on the LabVIEW interface to align the ultrasound and camera position measurements. The path to follow was designed as a twisted U-shape. The ultrasound measurements were in good agreement with the measurements using cameras. The RMS error between camera measurements and ultra-sound was 3.41±2.48 mm (1.88±1.65 mm about the x axis, 1.08±0.8 mm about the y axis, and 2.16±2.25 mm about the z axis).

The closed loop control was configured to follow the above twisted U-shape loop. The swimmer started from the bottom of the workspace. The swimmer first swam vertically upward until it was detected by the ultrasound device. Closed-loop control started automatically after the first detection.

The system may be remotely controlled by a qualified surgeon. Remote accessibility will enable the MRS to be used in many different communities, including small hospitals where a qualified surgeon is unavailable. The use of ultrasound, a technology widely available within clinics, paired with the presented control method, will reduce the patient's exposure to X-rays in tracking the swimmer and decrease the risk of cancer.

Although relative terms such as “outer,” “inner,” “upper,” “lower,” and similar terms have been used herein to describe a spatial relationship of one element to another, it is understood that these terms are intended to encompass different orientations of the various elements and components in addition to the orientation depicted in the figures. Furthermore, as used herein, the terms “connect,” “connection,” “connected,” “in connection with,” and “connecting” may be used to mean in direct connection with or in connection with via one or more elements. Similarly, the terms “couple,” “coupling,” and “coupled” may be used to mean directly coupled or coupled via one or more elements. The terms “substantially,” “approximately,” “generally,” and “about” are defined as largely but not necessarily wholly what is specified (and includes what is specified; e.g., substantially 90 degrees includes 90 degrees and substantially parallel includes parallel), as understood by a person of ordinary skill in the art. The extent to which the description may vary will depend on how great a change can be instituted and still have a person of ordinary skill in the art recognized the modified feature as still having the required characteristics and capabilities of the unmodified feature.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a,” “an” and other singular terms are intended to include the plural forms thereof unless specifically excluded.

What is claimed is:

1. A system for 3D navigation of a magnetic rotating swimmer (MRS), the system comprising:
a magnetic manipulator comprising coils defining a 3D workspace, the magnetic manipular is operable to steer the MRS in the 3D workspace;
a probe positioned to obtain a 2D image of an imaging plane from the 3D workspace;
a robotic arm coupled to the probe and operable to move the probe and the imaging plane; and
a processor network in communication with the magnetic manipulator, the probe, and the robotic arm, the processor network comprising instructions:
to operate the magnetic manipulator to move the MRS on a path in the 3D workspace;
to obtain 2D images with the probe while the MRS is moving in the 3D workspace;
to detect the MRS in the images; and
to start, in response to detecting the MRS, closed-loop control of the robotic arm to move the probe to track the MRS movement in the 3D workspace.

2. The system of claim 1, wherein the robotic arm has six degrees of freedom.

3. The system of claim 1, wherein the probe is an ultrasound probe.

4. The system of claim 1, wherein the closed-loop control consists of data from the 2D image.

5. The system of claim 1, wherein the processor network includes an instruction to change a reference frame of the MRS from coordinates in the imaging plane to coordinates in the 3D workspace.

6. The system of claim 1, wherein the processor network includes an instruction to move the robotic arm to position the imaging plane on a path point closest to a last known position of the MRS and parallel to the path at the path point.

7. The system of claim 1, wherein the probe is an ultrasound probe; and
the closed-loop control consists of data from the 2D image.

8. The system of claim 7, wherein the processor network includes an instruction to change a reference frame of the MRS from coordinates in the imaging plane to coordinates in the 3D workspace.

9. The system of claim 7, wherein the processor network includes an instruction to move the robotic arm to position the imaging plane on a path point closest to a last known position of the MRS and parallel to the path at the path point.

10. The system of claim 7, wherein the processor network includes instructions:
to move the robotic arm to position the imaging plane on a path point closest to a last known position of the MRS and parallel to the path at the path point; and
to change a reference frame of the MRS from coordinates in the imaging plane to coordinates in the 3D workspace.

11. A method for 3D navigation of a magnetic rotating swimmer (MRS) in a 3D workspace, the method comprising:
positioning the MRS in a 3D workspace defined by coils of a magnetic manipulator;
positioning a probe coupled to a robotic arm to obtain a 2D image from the 3D workspace along an imaging plane;
navigating the MRS along a 3D path in the 3D workspace with the magnetic manipulator;
obtaining the 2D image along the imaging plane;
processing the 2D image to detect the MRS in the imaging plane; and
initiating, in response to detecting the MRS in the imaging plane, a closed-loop control of the robotic arm to move the probe to track the MRS movement along the 3D path.

12. The method of claim 11, wherein the MRS is an untethered millimeter scale device.

13. The method of claim 11, wherein the closed-loop control consists of data from the 2D image.

14. The method of claim 11, wherein the probe is an ultrasound probe.

15. The method of claim 11, wherein the closed-loop control comprises changing a reference frame of the MRS from coordinates in the imaging plane to coordinates in the 3D workspace.

16. The method of claim 11, wherein the closed-loop control comprises manipulating the robotic arm and positioning the imaging plane on a path point closest to a last known position of the MRS and parallel to the path at the path point.

17. The method of claim 11, wherein:
the probe is an ultrasound probe; and
the closed-loop control comprises changing a reference frame of the MRS from coordinates in the imaging plane to coordinates in the 3D workspace.

18. The method of claim 17, wherein the closed-loop control comprises manipulating the robotic arm to position the imaging plane on a path point closest to a last known position of the MRS and parallel to the path at the path point.

19. The method of claim 18, wherein the MRS is an untethered millimeter scale device.

20. The method of claim 17, wherein the closed-loop control consists of data from the 2D image.
